(19) United States
(12) Patent Application Publication
(10) Pub. No.: US 2010/0161067 A1
(43) Pub. Date: Jun. 24, 2010

(54) KNEE PROSTHESIS

(75) Inventors: KHALED J. SALEH, Springfield, IL (US); William Mihalko, Germantown, TN (US); Said Moussa, Chamarandes (FR); Dominique Mouillet, Semoutiers (FR)

Correspondence Address: RATNERPRESTIA, P.O. BOX 980, VALLEY FORGE, PA, 19482, US

(73) Assignee: AESCULAP AG, TUTTLINGEN (DE)

(21) Appl. No.: 12/484,594

(22) Filed: Jun. 15, 2009

Related U.S. Application Data
(60) Provisional application No. 61/140,183, filed on Dec. 23, 2008.

(51) Int. Cl. A61F 2/38

(52) U.S. Cl. 623/20.31

(57) ABSTRACT

A knee prosthesis having a femoral component with two condyles and an opening disposed between the two condyles. The prosthesis includes a cam extending between the condyles forming a posterior boundary to the opening. Also included is a tibial component having bearing surfaces to support each of the femoral component condyles, and a post disposed between the bearing surfaces and extending superior from the tibial component. The femoral component and tibial component are engageable by contact between the femoral condyles and tibial bearing surfaces, and by contact between the cam and post during at least a portion of flexion between the femoral and tibial components. Moving the femoral and tibial components in flexion from about 45° to about 145° causes a contact region between the cam and post to move inferiorly from a first contact region to a second contact region while also causing rotation between the tibial and femoral components.

This application claims the benefit of earlier filed U.S. Provisional Application Ser. No. 61/140,183 filed Dec. 23, 2008. It is hereby fully incorporated by reference as if fully set forth herein.

### FIELD OF INVENTION

The field of invention relates to artificial joints, and more particularly to knee prostheses.

### BACKGROUND

As is the case with many joint prostheses or replacements, replicating natural anatomical movement through artificial mechanical devices proves challenging. This is true especially with the knee, which allows for relative complex movement and kinematics between the femoral condyles and the tibia. This relative motion is complex in that it accounts for both rolling and sliding between the contact surfaces at varying rates throughout the flexion arc. Along with such movement during knee bending is a rotational movement between the tibia and femur. As such, knee prostheses have historically tried to replicate the full range of knee movement, throughout and between full flexion and extension in all planes (coronal-varus/valgus, sagittal-flexion, transverse-rotation). True anatomical movement would allow rollback and translation of the femoral condyles on the tibia, all while also allowing rotational movement during flexion/extension.

Prior art designs have included femoral components with cams and tibial components with posts. It has been disclosed that an asymmetrical cam can be utilized to cause rotation between the two components. These designs, however, have taught architectures that require relatively high posts to support upward movement of the cam during flexion.

### SUMMARY OF THE INVENTION

The present invention provides a knee prosthesis having a femoral component with two condyles with an opening disposed between the two condyles, and a cam extending between the condyles forming a posterior boundary to the opening. Also included is a tibial component having bearing surfaces to support each of the femoral component condyles, and a post disposed between the bearing surfaces and extending superior from the tibial component. The femoral component and tibial component are engageable by contact between the femoral condyles and tibial bearing surfaces, and by contact between the cam and post during at least a portion of flexion between the femoral and tibial components. By moving the femoral and tibial components in flexion from about 45° to about 145°, the contact region between the cam and post moves inferiorly from a first contact region downward to a second contact region while also causing rotation between the tibial and femoral components.

### BRIEF DESCRIPTION OF THE FIGURES

FIG. 1 illustrates a tibial component in accordance with the present invention;

FIG. 2 illustrates a femoral component in accordance with the present invention;

FIG. 3 illustrates a tibial component (with a stem) and a femoral component mated in accordance with the present invention;

FIG. 4 illustrates a partial cross sectional view of the prosthesis of the present invention at about 0° flexion;

FIG. 5 illustrates a partial cross sectional view of the prosthesis of the present invention at about 90° flexion;

FIG. 6 illustrates a partial cross sectional view of the prosthesis of the present invention at about 145° flexion;

FIG. 7 illustrates a series of cross sectional views at three planes of interaction of the cam and post in accordance with the present invention;

FIG. 8 illustrates a partial cross sectional view of the prosthesis of the present invention at about full flexion;

FIG. 9 illustrates that of FIG. 8 but with the addition of a patellar implant;

FIG. 10 illustrates a top down view of the prosthesis of the present invention at about 45° flexion;

FIG. 11 illustrates a partial cross sectional view from the top down of the prosthesis of the present invention at about 90° flexion;

FIG. 12 illustrates a partial cross sectional view from the top down of the prosthesis of the present invention at about 145° flexion;

FIG. 13 illustrates a top down view of the prosthesis of the present invention at about 145° flexion; and

FIG. 14 illustrates a cross sectional view at high flexion showing separation of the lateral condyle from tibial component.

### DETAILED DESCRIPTION OF THE INVENTION

The present invention provides a knee prosthesis which allows for anatomically correct knee movement. It does so by providing an upper, or femoral, component which is designed to mechanically interact with a lower, or tibial, component to achieve kinematic movement consistent with a natural knee joint. Generally, the two pieces interact by providing several different contact surfaces, not all of which are engaged between the two components of the knee throughout the range of motion.

Two such contact surfaces are the load bearing condylar surfaces between the femoral component and the tibial component. These surfaces are defined by medial and lateral condylar surfaces which are referred to as the load bearing surfaces for a given knee joint. Specifically, a medial load bearing surface is defined between the medial femoral condyle and its counterpart on the tibial component, namely a medial tibial accommodating surface. Likewise, a lateral load bearing surface is defined between the lateral femoral condyle and its counterpart on the tibial component, namely a lateral tibial accommodating surface.

A different contact surface also exists, however, to cause rotational movement between the femoral and tibial components, during certain degrees of knee extension/flexion which will allow for a kinematic pattern that more closely resembles that of the natural knee. This contact surface is defined by interaction between a post on the tibial component (preferably polyethylene) and a cam surface on the femoral component (preferably metallic). Because the point of contact between the femoral condyles and their corresponding tibial load-receiving components changes in an anterior/posterior direction (that is to say there is front/back translation of the point of contact) during knee movement, the post and cam do not interact during all degrees of knee flexion. Instead, the post and cam only interact during those points of knee movement for which they are designed to cause a replicated natural knee kinematic envelop. This interaction occurs when the anterior/posterior movement of the femoral/tibial contact causes the post and cam to engage, or when flexion of the knee causes enough rollback of the femoral component to engage the tibial post against the cam of the femoral component.

It should be noted, however, that once flexion typically reaches about 45°, anterior/posterior translation does not stop but occurs at different rates in the medial and lateral compartments of the knee. Moreover, as the knee bends, the lateral condyle rolls back to a position of about 10-15 mm posterior at about 120° flexion, but the medial condyle rolls back only about 4-5 mm to a final position of about 1-3 mm posterior. This difference in posterior movement in the two compartments of the knee is seen as rotation of the femoral component on the tibial component, and occurs with continued rollback of the femoral condyles. This interaction of the post and cam, as well as the movement of the femoral condyles with respect to the tibial bearing surfaces will be addressed below.

The movement described is achieved through the present invention's architecture of the both the femoral component, the tibial component, and in particular the cam and post dimensions. All of these aspects are integrated into a system which provides for sophisticated, anatomical movement within the prosthetic knee of the present invention.

FIG. 1 shows a tibial component 100 in accordance with the present invention. This tibial component 100 has two load bearing surfaces, shown as load bearing surface 101 and load bearing surface 102. For a right knee joint, load bearing surface 101 would be the lateral condyle load bearing surface, and load bearing surface 102 would be the medial condyle load bearing surface. Post 110 is shown extending upward, or in a superior direction, from the lateral plane generally defining the tibial insert. Post 110 will be described in more detail below.

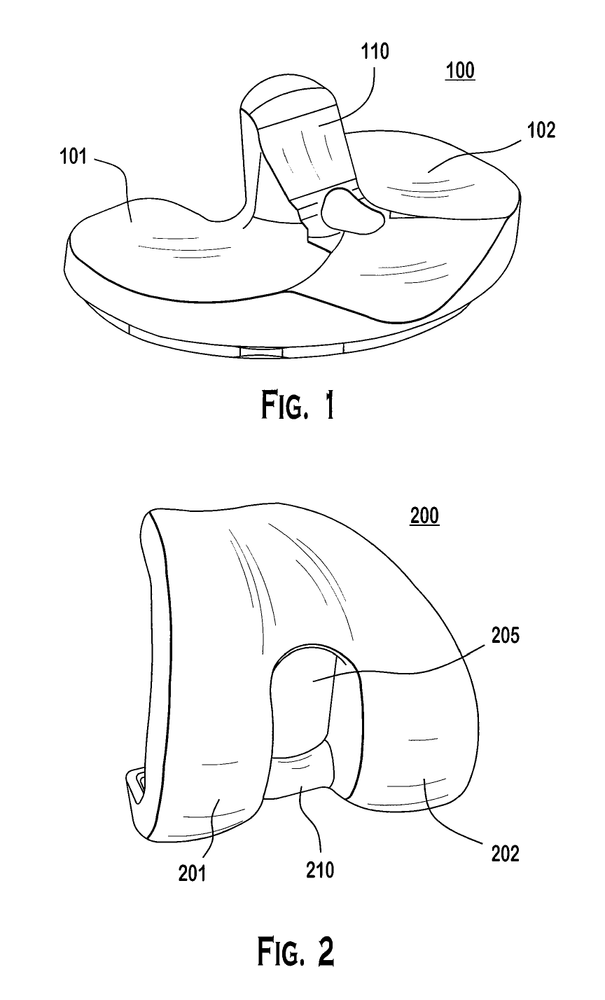

FIG. 2 illustrates a femoral component 200 in accordance with the present invention. Cam 210 is shown bridging a gap between the femoral condyles 201 and 202. Opening 205 is defined by the condyles 201 and 202 which extend anteriorly around the side of the opening opposite cam 210. Cam 210 is generally disposed in a posterior portion of the opening in the femoral component. Cam 210 and its dimensions will be defined in more detail below.

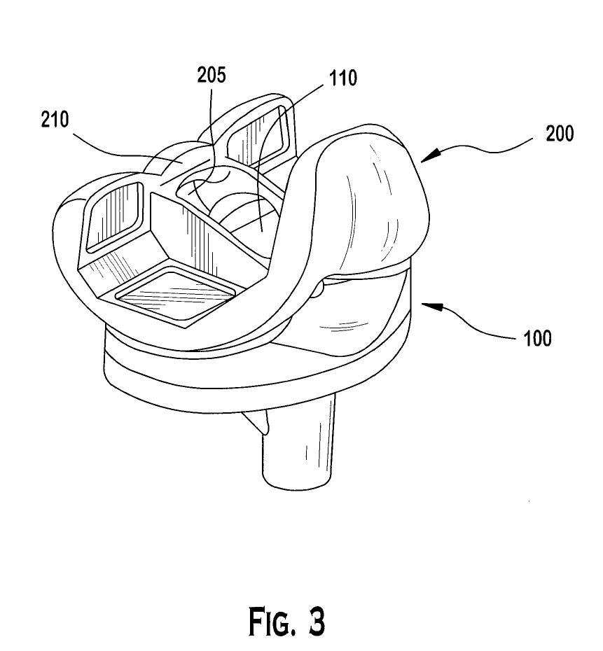

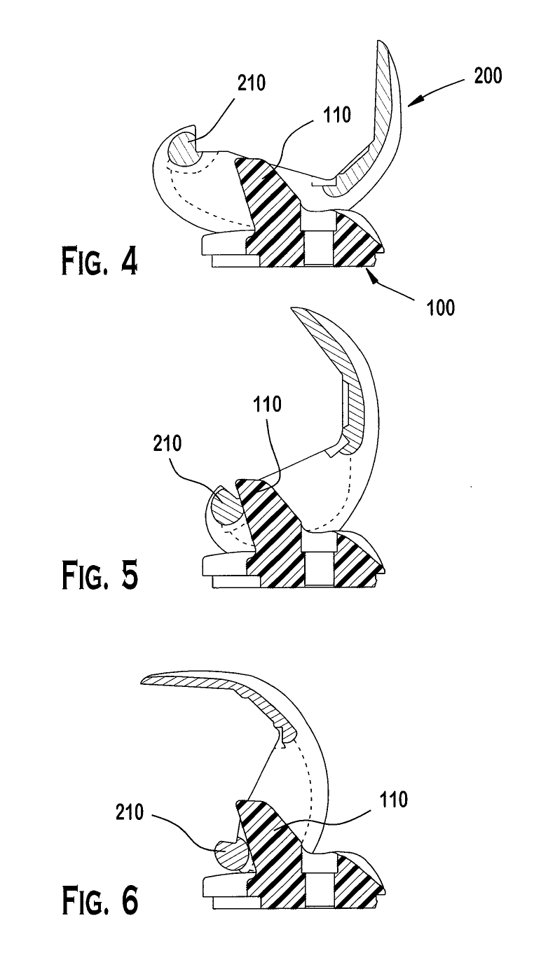

FIG. 3 shows femoral component 200 disposed atop tibial component 100. Post 110 is shown extending through opening 205. FIG. 3 shows the components in a position of 0° flexion. As can be seen from FIG. 3, cam 210 is not in contact with post 110 at this point. It is also noteworthy that in this position, there is no contact between the anterior surface of post 110 and the anterior boundary of opening 205. This aspect can be seen perhaps more clearly in FIG. 4, which shows a partial cross sectional view of that shown in FIG. 3. This aspect of the present invention is important because it reduces wear on the tibial post 110.

For an example of an implant having both anterior and posterior cams, see U.S. Pat. No. 6,325,828, which illustrates a femoral component having a blind hole or slot/recess (as opposed to an opening) bordered by cams on both sides (anterior and posterior). As such, and as explicitly disclosed, the anterior cam engages the post at full extension (or 0° flexion).

As the knee bends toward a flexion of about 45°, cam 210 moves toward post 110 as anterior translation occurs between the contact region of the femoral condyles and their respective load bearing surfaces on tibial component 100. The orientation of the two components, and in particular the cam and post, at 45° flexion, is illustrated in FIG. 5, which shows a partial cross sectional view of the components at about 45° flexion. At this point in the knee movement, the cam 210 has contacted post 110 and as further flexion occurs, the rotational movement caused by the interaction of the post and cam causes slight medial rotation of the femoral component with respect to the tibial component.

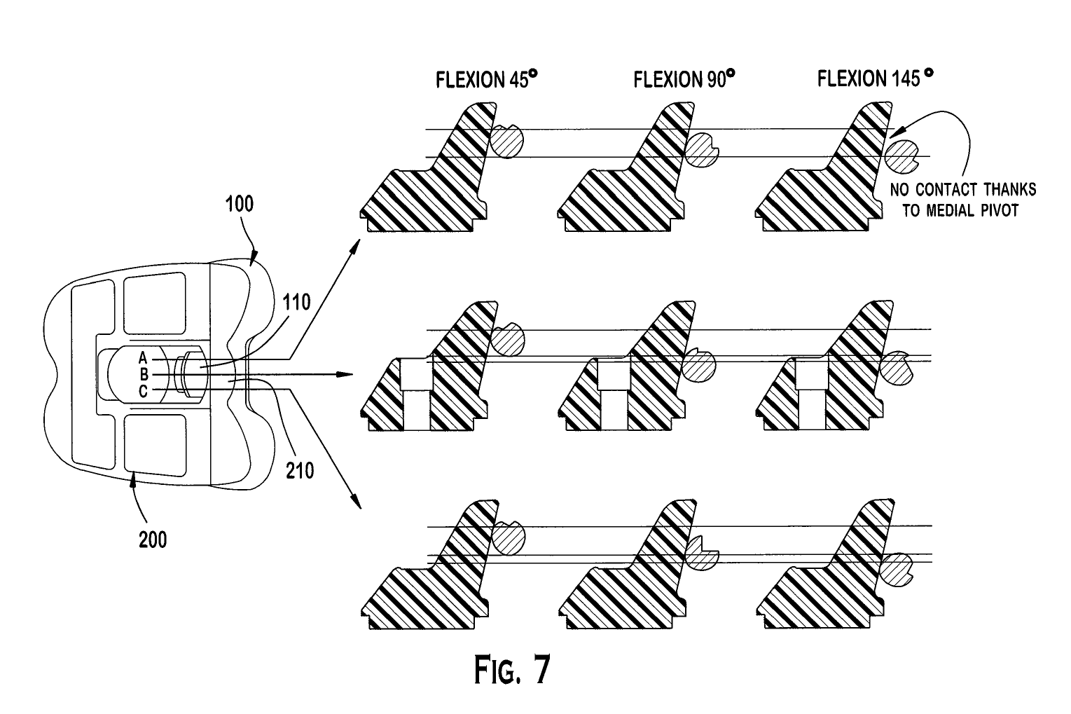

FIG. 6 shows the partial cross section of the two components after further knee flexion. Note that the contact point between the cam and post moves downward along the post, or inferiorly, as flexion increases. This is due to the architecture of the cam and post and is designed as a part of the knee movement based on the anatomical requirements of the natural knee joint.

Further defining this aspect of the invention is FIG. 7. FIG. 7 shows the cross sections of three different planes at three different angles of flexion. Planes A, B, and C are shown and illustrate the asymmetry of the cam 210 and the effect of that asymmetry on the rotation and inferior movement of the cam down the post as flexion increases. At 45° flexion, plane A indicates contact of the cam and post at a point relatively high on the post. As flexion increases to 90°, the cam is working its way down the post as the femoral component rotates medially with respect to the tibial component. At 145° flexion, not only has the cam moved further downward along the posterior side of the post (at planes B and C), but in fact, at plane A, or the lateral side of the cam, the cam has disengaged the post altogether as medial rotation has separated the cam from the post at this point. Thus, there is seen a medial rotation consistent with natural knee movement while the cam has actually moved down along the post. Stability is one advantage of the implant designed this way in accordance with the invention.

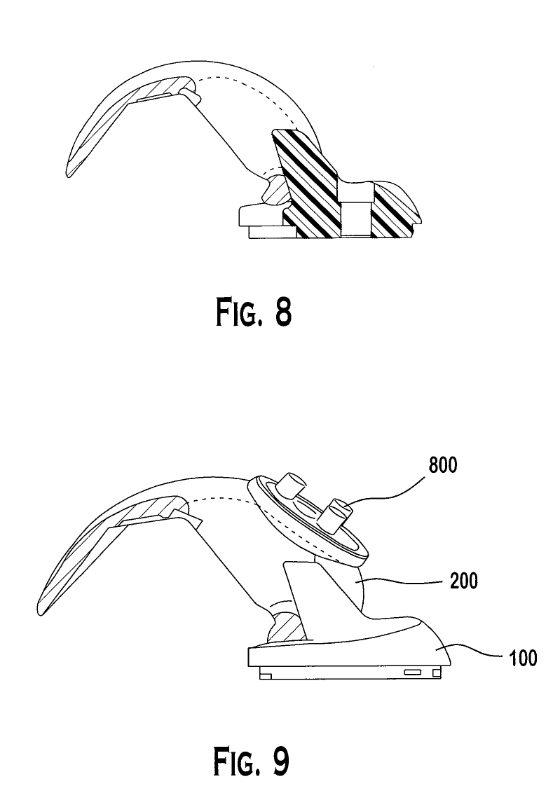

This later point is important to achieve natural knee movement with respect to a patellar implant. FIG. 8 shows knee prosthesis of the present invention at about 145° flexion. At this point, and as noted above, the cam has moved downward along the post. The post therefore only needs to be as high as is necessary to engage the cam at the first point of contact, namely at about 45° flexion (because after that the cam moves downward).

The relative shortness of the post is important because it allows for clearance of the patellar implant as shown in FIG. 9. There, patellar implant 800 is shown disposed on femoral component 200. Unlike prior art designs that have upward cam movement during flexion, and therefore require higher posts to extend upward from the initial point of contact, the present invention is configured to provide downward cam movement and therefore relatively shorter posts are necessary. This allows for patellar clearance during knee rotation as shown in FIG. 9.

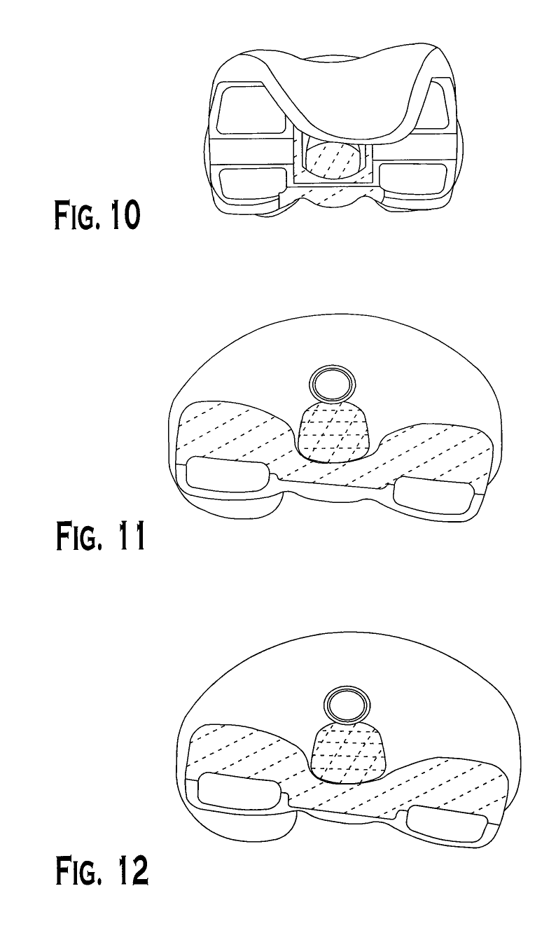

By way of further illustration, FIGS. 10-12 shows a top-down partial cross sectional view of the prosthesis during flexion of 45°, 90°, and 145°, respectively. As can be seen from these views, the cam has a shape and size quite different on the lateral side than on its medial side. This cam and its particular shape and orientation provides an angled surface which acts with the post to drive a very precise medial pivot and femoral rotation in the transverse plane.

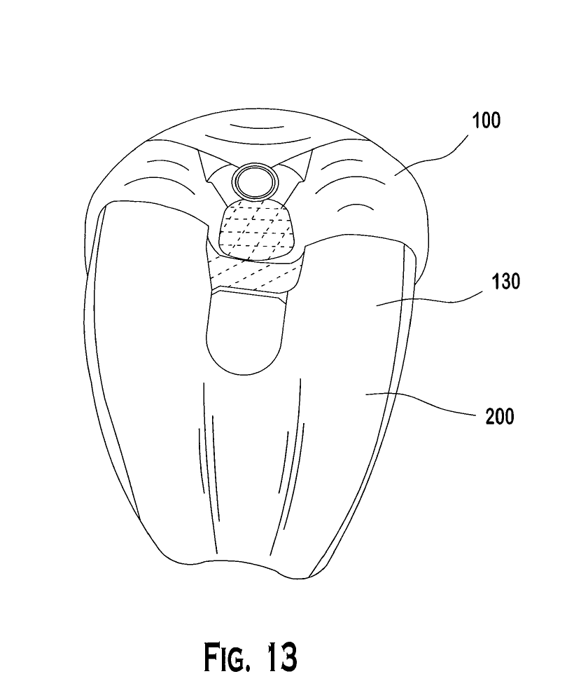

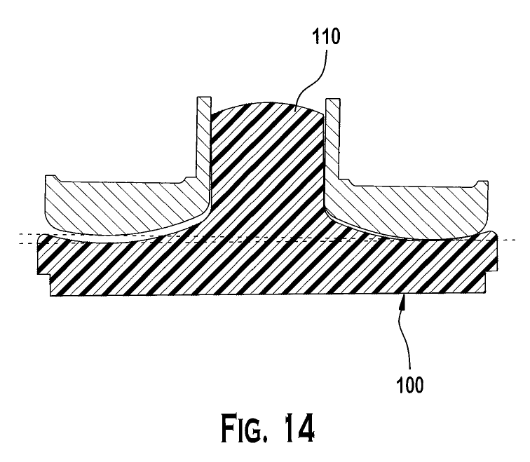

FIG. 13 illustrates a top down view of the cam and post interaction and also shows the medial rotation of the femoral component with respect to its tibial component. Note that even at this relatively high flexion, the cam is disposed somewhat under the post and enlarges in cross sectional area toward the lateral end of the cam where it abuts the lateral condyle 130.

It is also noteworthy that the design of the present invention provides for lift-off of the lateral condyle from the tibial load bearing surface at high flexion. See, for example, FIG. 14, which shows separation of the lateral condyle 140 from tibial component 100. This separation is due in part to the architecture of the cam and the post to which it engages during flexion. The separation so achieved aids in replicating anatomically correct movement.

One advantage to the prosthesis of the present invention is that it allows for less soft tissue strain by allowing for more anatomical movement instead of equal rollback in both compartments of the tibial insert. This design gives three advantages over previous designs: 1) less soft tissue strain due to more anatomical movement, 2) better natural motion replication in the medial compartment without increasing constraint, and 3) decreased tibial strain with no edge loading in the medial compartment. Although the above illustrations show knee flexion at 0°, 90°, and 145°, the range of motion allowed for in the design would be at least −10° (hyperextension) to about 160° (high flexion) with supported articulation in the medial and lateral compartments of the knee.

Moreover, as flexion continues beyond 45°, anterior/posterior translation continues to occur, but is guided by the post/asymmetric-cam interaction. Because of the relative dimensions of the post, and in particular the type of asymmetrical cam on the femoral component, proper rotational movement between the femoral component and tibial component is achieved.

Consistent with that described above, the interaction between the tibial component post and the femoral component tapered asymmetric cam, is designed to preferably begin at 45° flexion. It should be noted that the interaction can be controlled through manipulation of the dimensions of the post and cam. This is accomplished through varying the cross-sectional dimensions of the cam from a medial to lateral direction, with the lateral portion of the cam being generally larger than the medial portion. More specifically, the largest cross-sectional area of the cam occurs where the cam meets the lateral condyle. Moving in a medial direction, the cam tapers in a manner consistent with that which causes kinematic rotation as the knee bends past 45° flexion.

It is also noteworthy that there is no interaction between the post and cam at full extension. This prevents unnecessary wear on the tibial post which would otherwise weaken it over time and could even result in failure (i.e, it could shear off).

What is claimed:

1. A knee prosthesis comprising:
a femoral component having two condyles with an opening disposed between the two condyles, and a cam extending between the condyles forming a posterior boundary to the opening; and
a tibial component having bearing surfaces to support each of the femoral component condyles, and a post disposed between the bearing surfaces and extending superior from the tibial component;
the femoral component and tibial component engageable by contact between the femoral condyles and tibial bearing surfaces, and by contact between the cam and post during at least a portion of flexion between the femoral and tibial components;
such that moving the femoral and tibial components in flexion from about 45° to about 145°, causes a contact region between the cam and post to move inferiorly from a first contact region to a second contact region while also causing rotation between the tibial and femoral components.

2. The prosthesis of claim 1, wherein the cam has an asymmetrical cross section between its lateral end and medial end.

3. The prosthesis of claim 1, wherein the cam has an asymmetrical cross section between a lateral end region and a medial end region, and the lateral end region has a larger cross sectional area as compared to its medial end region.

4. A knee prosthesis comprising:
a femoral component having two condyles with an opening disposed between the two condyles, and a cam extending medially between the condyles and forming a posterior side to the opening, the cam having a larger cross sectional area in its lateral region as compared to its medial region; and
a tibial component having bearing surfaces to support each of the femoral component condyles, and a post disposed between the bearing surfaces and extending superior from the tibial component;
the femoral component and tibial component engageable by contact between the femoral condyles and tibial bearing surfaces, and by contact between the cam and post during at least a portion of flexion between the femoral and tibial components;
such that moving the femoral and tibial components in flexion from about 45° to about 145°, moves the lateral region of the cam away from the post such that there is no contact between the cam and post at the lateral region of the cam at flexion of about 145°.
